(19) United States
(12) Patent Application Publication
(10) Pub. No.: US 2022/0118202 A1
(43) Pub. Date: Apr. 21, 2022

(54) Airway cleansing, allergy treatment and agent delivery method and apparatus

(71) Applicant: Anthony David Wondka, San Ramon, CA (US)

(72) Inventor: Anthony David Wondka, San Ramon, CA (US)

(21) Appl. No.: 17/503,250

(22) Filed: Oct. 15, 2021

Related U.S. Application Data
(60) Provisional application No. 63/092,498, filed on Oct. 15, 2020.

(51) Int. Cl. A61M 15/08; A61K 9/12; A61P 11/02

(52) U.S. Cl. CPC A61M 15/08; A61K 9/0043; A61P 11/02; A61K 9/12

(57) ABSTRACT

A gas delivery device is described for applying gas to an airway, typically the nasal cavity, to cleanse the mucosa to alleviate symptoms of allergy, congestion, and other ailments related to the anatomy of the nasal cavity and surrounding anatomical structures. The device and method are advantageously a zero-mess treatment, can be self-applied by the user, and can be used with a naturally occurring, benign gas, therefore making it a safe, natural treatment without undesirable pharmacological side effects.

### CROSS-REFERENCE TO RELATED APPLICATIONS

This application claims benefit to U.S. Provisional Application No. 63/092,498 filed Oct. 15, 2020, the disclosures of which are hereby incorporated by reference in their entirety.

### BACKGROUND

Up to 30 percent of people in the world suffer from respiratory allergies, including hay fever, allergic rhinitis, and including symptoms such as running nose, congested nose, sneezing, itchy eyes, itchy palate, itchy nose, itchy throat, coughing, itchy skin, and other symptoms. This problem costs the US economy 150 billion dollars in lost productivity per year. Allergy symptoms are typically treated by drugs, which have side effects. For example, diphenhydramine can cause drowsiness, fatigue, tiredness, sleepiness, dizziness, disturbed coordination, constipation, dry mouth and other effects, and pregnant or breastfeeding women may not be good candidates for this drug. Some non-pharmacological treatments, such as saline, water or steam rinsing of the nasal cavity, are available as options to those who cannot tolerate or chose to not use drug treatments. However, these treatments are less effective than drugs and are messy, therefore significantly limiting their use. An alternative treatment, the application of CO2 gas into the nasal cavity, has been reported in the literature, which solves the problems of drug-related side effects, and messy use.

### SUMMARY

The device and method described in this invention improve on the state-of-the-art of non-drug, mess-free, allergy treatments. An apparatus delivers a biocompatible gas into the nasal cavity mucosal tissues, where it interacts with cilia and cells in the epithelium to cleanse them of allergens and allergy-causing particles. The gas is direct acting on the ciliary surfaces and the surfaces and membranes of the cells, and through the interstices of the cells. Once the allergens are washed away from these structures, the allergic response can be avoided or curtailed. In a first embodiment, a gas delivery device is used which includes a replaceable compressed gas cartridge, a gas flow regulating and metering mechanism, and on/off mechanism, and a gas delivery nosepiece. In a second embodiment, a gas delivery device includes gas delivery nosepiece centering cushion that seals the nose and prevents the delivery nosepiece from touching the mucosa and getting soiled. In a third embodiment, a reservoir is used to receive the gas from the compressed gas source, and an injection mechanism is used to inject the gas. In a fourth embodiment a pump is used to inject the gas into a nostril from a reservoir and optionally a vacuum pump is used to remove gas from the contralateral nostril, creating a flow loop through the nasal cavity from and back to the device. In a fifth embodiment an external gas source is used. In a sixth embodiment, the device's gaseous delivery is used as a carrier agent to deliver medicine. In a seventh embodiment the device's primary gas that is used for the primary treatment, entrains a secondary agent from a cartridge to create a secondary beneficial effect, secondary to the primary effect of the primary gas. In a seventh embodiment, the gas is inhaled into the lung for bronchial, lung, or systemic treatment. In many embodiments, the gas of choice is CO2 gas, and in some embodiments, the gas can be O2 gas, or N2 gas, or combinations of CO2, O2 and N2. CO2 gas has the advantage of being biocompatible and compressible to its liquid state such that large gas volumes can be stored in a small volume container in its liquid state. Beverage grade CO2 which is 95.95% pure CO2 is safe and effective for the purposes of this invention. Other gases may be used.

### BRIEF DESCRIPTION OF THE DRAWINGS

FIG. 1 is a front-line view of the gas delivery apparatus.

FIG. 2 is a top sectional view of the cross-section A-A of Figure, enlarged for legibility.

FIG. 3 is an exploded hidden line view of the apparatus of FIG. 1.

FIG. 4 is a front-line view of the apparatus of FIG. 1 with a first alternative delivery nozzle assembly.

FIG. 5 is a front-line view of the apparatus of FIG. 1 with a second alternative delivery nozzle assembly.

FIG. 6 is a cut away side view of a nostril of a person, with a side line view of the apparatus inserted into the nostril and delivering the gas.

FIG. 7 is a front-line drawing view of a fourth alternative delivery nozzle assembly.

FIG. 8 is a front schematic view of an alternate apparatus with a gas reservoir and injection mechanism.

FIG. 9 is a pneumatic schematic diagram of the apparatus shown in FIG. 8.

FIG. 10 is a front schematic view of an alternate apparatus with a gas pump to inject gas into a nostril, and a vacuum pump to remove gas from the contralateral nostril, creating a circulation flow path.

FIG. 11 is a pneumatic schematic diagram of the apparatus in FIG. 10.

FIG. 12 is a front-line drawing view of an alternate apparatus with an external gas supply, with gas delivery pump and return path vacuum pump.

FIG. 13 is a front-line drawing view of an alternative apparatus using the removable gas delivery nozzle assembly shown in FIG. 7 and with a secondary substance delivery feature.

FIG. 14 is a front partial hidden line drawing view of an alternative apparatus with a dual nozzle gas delivery nozzle assembly and with multiple fixed orifices and on/off buttons.

FIG. 15 is a front line partial hidden line view of an alternative gas delivery device with a gas refill feature, and a porous shield to protect the porous gas delivery nozzle.

### DETAILED DESCRIPTION OF THE EMBODIMENTS

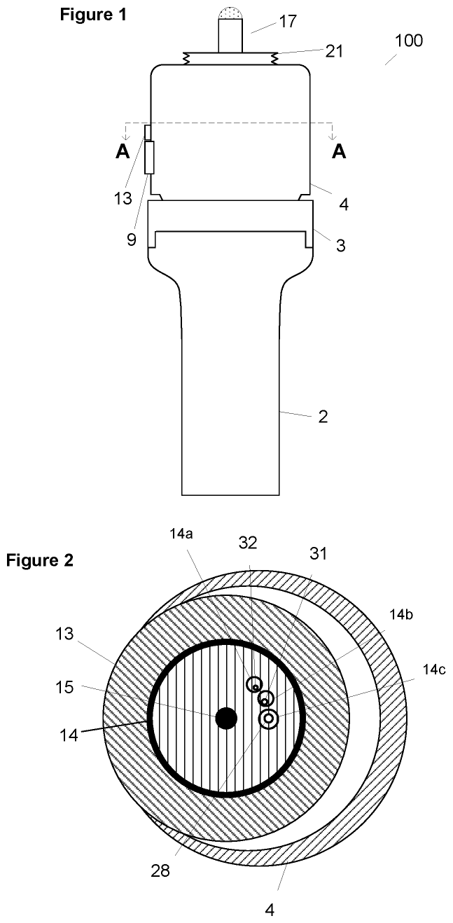

FIG. 1 describes a nasal gas delivery device 100. A gas cylinder is replace-ably housed in a bottom cylinder cartridge housing 2. To install the cylinder, the lower housing is removed from the top cylinder cartridge housing 3, typically by unscrewing a threaded connection between the two pieces, thus freeing the cylinder. When installing a new cylinder, it is placed in the lower housing and the housing is screwed into the top cylinder cartridge housing 3, which is secured in place to the delivery handpiece housing 4. Once a gas cylinder is installed, the on/off button 9 turns on and off the flow of gas from the cylinder, and a flow control ring 13 controls the flow rate and ejection pressure of the gas out of the nosepiece nozzle assembly 17. A nostril cushion 21 helps prevent the gas from seeping out of the nose, and provides a gentle cushioning when the apparatus is pressed against the nostril rim. In subsequent figures, the on-off control mechanisms will become clearer.

In FIG. 2 the flow control ring 13 is shown at section A-A of FIG. 1. The ring center is non-concentric with the center of the delivery handpiece housing 4. The ring includes multiple orifices of different diameters; a large orifice 28, a medium orifice 31 and a small orifice 32. More orifices may be included, or there may be just two orifices. The largest orifice will allow the highest pressure and flow to be emitted out of the device, and the smallest orifice will allow the lowest pressure and flow to be emitted out of the device. The user can set the orifice based on therapeutic results, comfort, and if the gas supply is low, increase the setting to a larger orifice to increase the dose if needed. The ring is rotated about an axle 15, which aligns the desired orifice with the gas flow channel in the apparatus above and below the ring, shown in later figures. Gas is sealed within the desired orifice setting with seals 14a, 14b, 14c surrounding each of the orifices. Other flow control mechanisms are contemplated and included within the present invention, such as a variable orifice that is controlled with a dial or with the on-off mechanism (not shown in the Figures for purpose of brevity).

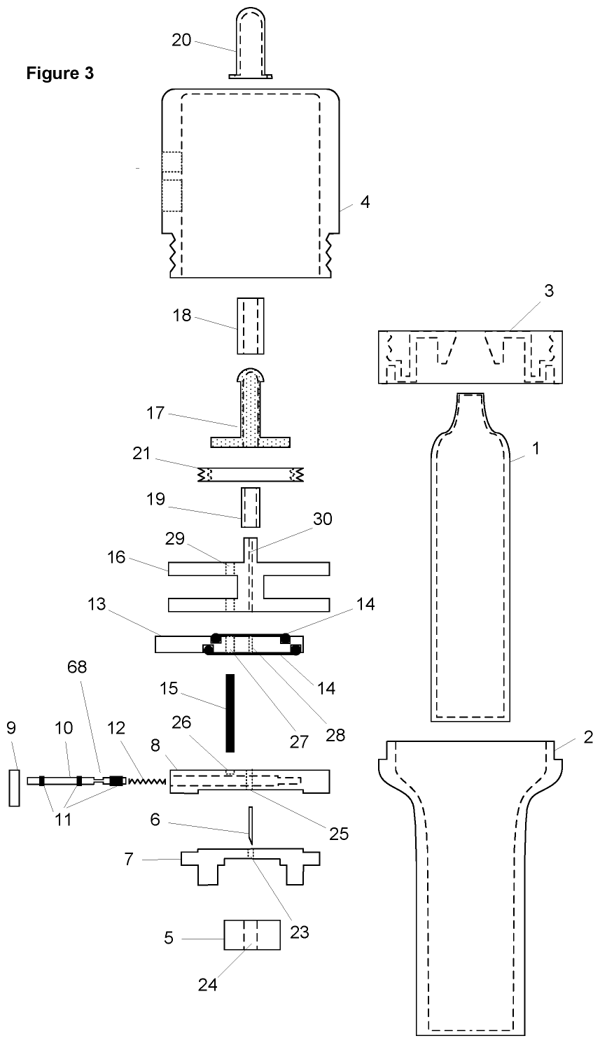

FIG. 3 is an exploded view of the apparatus shown in FIG. 1. The compressed gas cylinder 1 is placed into the bottom cylinder cartridge housing 2, which is then screwed into the top cylinder cartridge housing 3, and in doing so, the top of the gas cylinder 1 is pierced by the piercing needle 6. The top rim of the gas cylinder 1 is sealed by the cylinder rim seal 5, which is compressed against the cylinder's top rim when the bottom cylinder cartridge housing is screwed into the top cylinder cartridge housing. The piercing needle is firmly positioned by being pressed into the piercing needle seat 23 in the piercing needle holder 7. The cylinder rim seal 5 is situated into a pocket on the bottom of the needle holder 7 and includes a piercing needle channel 24 to accommodate the piercing needle. The on/off mechanism consists of an on/off button manifold 8, an on/off plunger 10 that slides in and out of the manifold 8, a plunger spring 12 which causes the gas flow state to be normally off, an on/off button 9 connected to the end of the plunger 10, lubricous plunger seals 11. When the plunger 10 is pressed into the manifold 8 the plunger notch 68 in the plunger aligns with the manifold flow channel 25 in the manifold, allowing gas to flow through the on/off mechanism. Otherwise, the spring 12 aligns the rightmost seal 11 with the manifold flow channel 25 to close the flow channel 25 and the gas flow is shut off. An axle 15, placed through a ring through hole 27, is used to position the flow control ring 13, and allows the ring to rotate within the handpiece housing 4 non-concentrically, with the channels through the ring additional sealed by ring seals 14. The axle is held in place at the bottom with a catch 26 in the on/off manifold 8 and at the top by through holes 29 in the axle hub and positioner 16. As previously described, an orifice in the ring 13, such as the large orifice 28, is aligned by rotating the ring so that the orifice is aligned with the flow channel 25 and it is also aligned with the flow channel 30 in the hub 16. The flow channel 30 is sealed with the hub seal 19, which seals the hub to the porous nosepiece 17. A sleeve 18 is placed around the nosepiece to direct the gas to flow out of the porous tip and not the sides, and to keep most of the nosepiece from getting soiled. A nostril cushion 21 is coupled to the delivery handpiece housing 4 and porous nosepiece 17 to cushion the nosepiece nozzle assembly against the nostril opening. The nosepiece nozzle assembly comprises the porous nosepiece 17, the sleeve 18 and the nostril cushion 21, maybe removable from the delivery handpiece housing 4. A shield 20 may be used to keep the nosepiece clean when not in use.

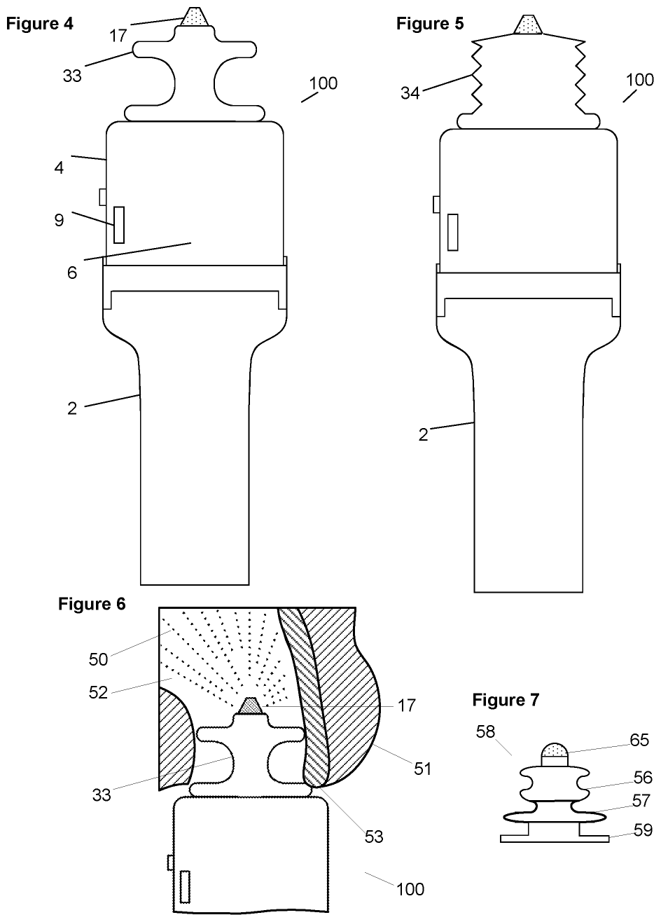

FIG. 4 describes an apparatus similar to that in FIG. 1, however with a centering and sealing cushion 33 which centers the porous nosepiece 17 in the user's nostril. FIG. 5 shows a centering and sealing pillow 34 which also accomplishes centering the nosepiece in the user's nostril. As shown in FIG. 6, centering the nosepiece has two primary advantages; first, it keeps the tip of the porous nosepiece 17 away from the walls of the nostril 51 so that the gas delivery profile is centered within the nasal cavity 52, and for example not directed to a wall of the nose. Secondly it prevents the porous nosepiece 17 from getting soiled if it were to come into contact with the walls of the nose. The centering and sealing pillow 34 also seals against the rim of the nostril 53 with the compliant flange at its base.

Now referring to FIG. 7, an alternative gas delivery nozzle assembly 58 is shown, which includes a centering ring 56 to center the delivery tip within the nostril, and an airway seal 57, the base of which seals against the outside rim of the nostril. Also, a nozzle assembly connector 59 may removably connect the delivery nozzle assembly to the remainder of the device. This delivery tip has the advantage of centering the porous gas delivery nozzle 65 in the center of the nostril, and sealing the nostril. When this device is used, the patient can exhale gently against the gas while the gas is being delivered, which will create a gentle positive pressure inside the nasal cavity, since the seal is sealing against the rim of the nostril, and help drive via pressure gradient the gas into the nasal mucosa and to the cellular structures where it will increase the cleansing effect due to the pressure.

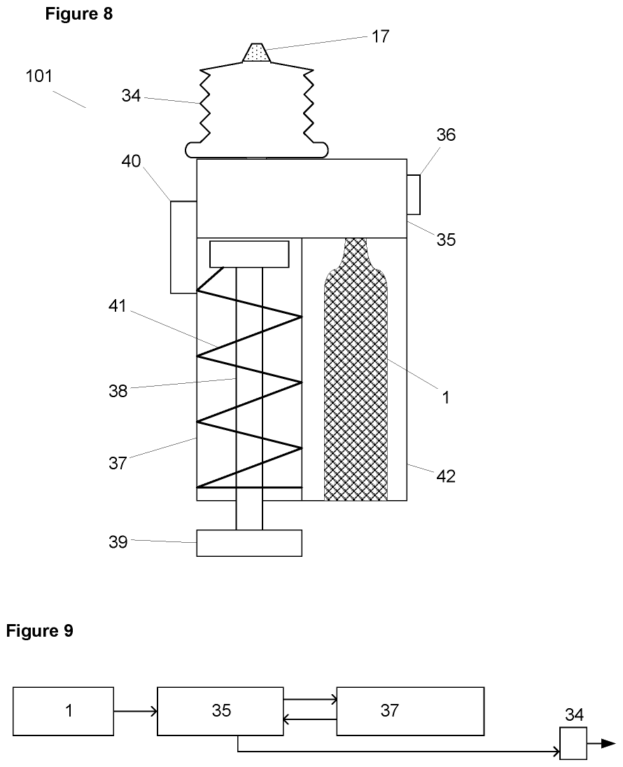

FIG. 8 describes an alternative apparatus 101 in which the gas cylinder 1 is housed in a cylinder cartridge 42. Rather than gas being delivered directly from the cylinder 1 to the porous nozzle 17, the gas is sent through a reservoir fill and delivery manifold 35 by actuation of the fill button 36, into an intermediary syringe reservoir 37 where a plunger spring 41 will be compressed with stored energy. From the reservoir, the gas is delivered to the user by actuation of the spring release button 40, which releases the stored energy of the spring allowing it to return to its resting state, which causes the syringe plunger 38 and handle 39 to force the gas out of the reservoir. FIG. 9 shows a pneumatic block diagram of the apparatus shown in FIG. 8.

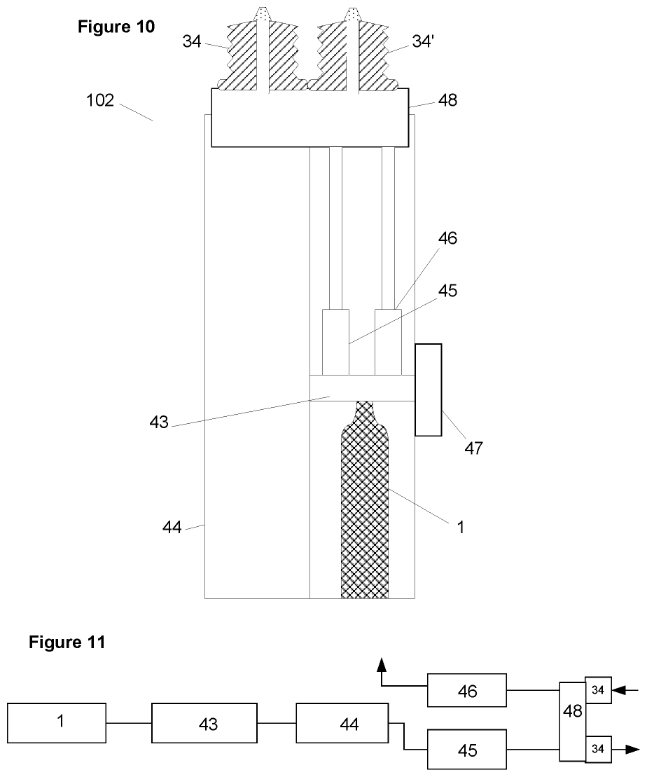

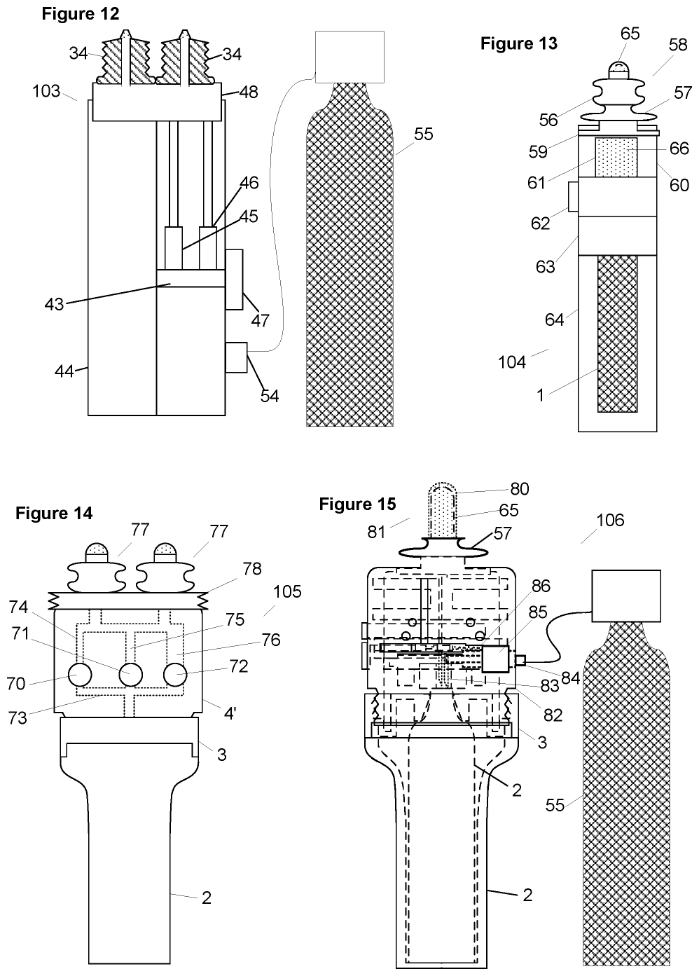

FIG. 10 describes an alternative apparatus 102 with dual nozzles 34 and 34′. This apparatus provides both gas delivery and active gas removal, by use of a pressure pump 45 and vacuum pump 46, respectively. Gas may be temporarily stored in the accumulator 44 which receives the gas through the accumulator fill and empty manifold 43 and accumulator manifold control button 47. The gas is delivered into the airway and drawn back out of the airway through a nosepiece assembly base 48 and the dual nozzles. This apparatus creates a circulation route for the gas, to help the gas travel through the nasal cavity by delivering it into one nostril and sucking it out in the other nostril. FIG. 11 is a pneumatic block diagram of the apparatus 102. FIG. 12 describes an alternative apparatus 103 which is similar to the apparatus shown in FIG. 10, with the main difference that the gas cylinder is an external cylinder 55 with regulator. The gas from the external cylinder's regulator fills the accumulator 22 through an inlet manifold 53.

FIG. 13 shows an alternative feature in which an additional therapeutic or wellness agent 66 is delivered when the primary gas is being delivered. Specifically, an agent cartridge 61 is placed inside the delivery device and is positioned in the primary gas flow path. When the primary gas passes the cartridge 61, it entrains the secondary agent 66. The cartridge 61 can be replaced within the cartridge housing 60. Gas flow is actuated by the actuator button 62, which opens the gas flow valve in the flow-control subassembly 63. The gas cylinder 1 can be replaced by removing the gas cartridge holder 64. The removable nosepiece nozzle assembly 58 is that shown in FIG. 7. The secondary agent may be a drug, a coagulant such as anhydrous aluminum sulfate to prevent the potential side effect of minor nose bleeding caused by the cleansing gas, an oil, a scenting agent or aroma agent, a lubricant, a moisturizer, a surface tension reduction agent to facilitate diffusion and penetration into the tissues, or other ingredient that provides a certain effect. The purpose of the secondary agent may be to cause a euphoric effect, a calming effect, an awakening effect, a sleepy effect, a mind clarity effect, antimicrobial or antibacterial or antiviral or antifungal effect, or other effect. Or, the purpose may be to cause an even more therapeutic effect such as a pharmacological effect. The purpose of the secondary agent may be to help tolerate the slight stinging of the primary gas delivery, or to prevent minor bleeding of the capillaries, or to help diffusion of the primary gas, or to have one of the effects previously noted. The secondary agent activity may also be anti-microbial, anti-viral, anti-bacterial, anti-fungal, a disinfectant, an anesthetic, an analgesic, a reversing agent for a poison or for a drug, an anti-inflammatory, a steroid, a neurotransmitter, a stimulant, a depressant, or caffeine. The secondary agent cartridge is typically replaceable, so the user can either refill it, or change the agent for the desired effect.

FIG. 14 shows a partial hidden line front view of an alternative gas delivery device 105. This device includes multiple on/off buttons and gas flow channels to control the dose of gas delivery. Gas flows through a universal channel 73 to multiple normally closed valves, controlled by buttons 70, 71, and 72, contained in a modified delivery handpiece housing 4′. The flow channel through each of the valves, controlled by the buttons regulates the delivery flow rate and pressure by virtue of the channel size. Flow channel 74 being a low dose, flow channel 75 being a medium dose, and flow channel 76 being a high dose. The device 105 also includes dual nosepiece delivery nozzles 77 in order to deliver the gas to both nostrils simultaneously. The structure of the nosepieces 77 allow them to rock about the nasal sealing cushion 78 in order to fit into a wide variety of nostril geometries.

FIG. 15 shows a front-line view of an alternative gas delivery system 106 which includes an alternative nosepiece nozzle assembly 81 and an internal gas cylinder refill mechanism. The nosepiece nozzle assembly includes a porous nozzle 65 covered by a porous shield 80. The porous shield prevents secretions from accumulating on the nozzle and blocking the gas output from the nozzle. The porous shield can be removed and cleaned frequently. It can be noticed in the various figures that many of the nosepiece nozzle assembly components are designed to be large enough to not be a choking hazard if they are inadvertently misassembled and come loose from the gas delivery device. The device 106 also has a gas refill system, which includes an alternative upper housing 82 that includes a refill needle 83, a refill conduit 86, a refill button 85 coupled to a normally closed refill valve (not shown), and a refill connector 84 to which the external gas cylinder and regulator 55 is attached to refill the internal cylinder 1.

The device may be configured to introduce the gas to the nose or to the mouth, or may be configured to deliver the gas slightly inside an airway opening, or configured to be delivered deeper into the airway, such as in the throat, larynx, and even in the tracheobronchial tree. Typically the nasal mucosa is treated, but the sinus and oral mucosa can also be treated. The invention takes into account mixing and combining different elements described in each of the figures, and for brevity, each of the conceived combinations are not described separately.

1. A delivery device for delivering a gaseous substance for cleansing airway structures, comprising (I) a gas delivery nozzle assembly, (ii) a gas delivery controller and (iii) a replaceable gas cartridge, wherein:
(1) the delivery nozzle assembly comprises:
a. a nozzle with base, midsection and tip, wherein the base and midsection are non-porous and the tip is porous,
b. a tip diameter of 0.125-0.250 inches,
c. a compliant centering ring at the midsection, to center the tip,
d. a compliant seal at the base to seal against the rim of the nose,
e. a removable connection at the base to removably connect to the delivery controller,
(2) the gas cartridge comprises:
a. a CO2 gas of a purity grade of 99.95% or better, compressed into its liquid state,
b. a mating connection mechanism to the gas delivery controller,
(3) the delivery controller comprises:
a. a normally closed gas flow port and a button to open the normally closed gas flow port;
b. a flow control mechanism to adjust the gas flow rate, the flow control mechanism consisting of (I) at least two orifices, a first orifice with a first size, and a second orifice with a second size, (ii) at least two settings, a first setting coupling the gas flow port with the first orifice size and a second setting coupling the gas flow port with the second orifice size, the first orifice size generating a first flow rate and the second orifice size generating a second flow rate;
c. a piercing needle to puncture and pneumatically communicate with the gas in the gas cartridge;
d. a seal to seal at the rim of a gas source cartridge;
e. a connection mechanism to removably connect the gas cartridge;
f. a removable porous shield to cover and protect the nosepiece from attracting occlusive substances;
g. a removable cap to protect the nosepiece and shield from debris when not in use, with an opening at its base and a substantially flat surface at its patient end.

2. A gas delivery device adapted to apply a flow of cleansing gas to an airway opening for the purpose of for cleansing airway tissues, the device comprising:
(1) a gas delivery nozzle;
(2) a compressed gas cylinder containing the cleansing gas;
(3) a reservoir to temporarily house the cleansing gas;
(4) a valve to transfer the gas from the cylinder to the reservoir;
(5) a mechanism to transfer the gas from the reservoir to the gas delivery nozzle, the mechanism comprising a pushing device and a valve;

3. A gas delivery device adapted to apply a flow of cleansing gas to an airway opening for the purpose of for cleansing airway tissues, the device comprising:
(1) a gas delivery nozzle;
(2) a compressed gas cylinder containing the cleansing gas;
(3) at least one flow channel, a first flow channel including an orifice of a first size, and a second flow channel including an orifice of a second size; the flow channels coupled to the gas delivery nozzle;
(4) a first button coupled to the first flow channel, and a second button coupled to the second flow channel, the buttons comprising an on state and a normally off state, the on state opening the flow channels and the off state closing the flow channels.

4. A device as in claim 1 comprising a removable cartridge containing a secondary agent, positioned in the flow path of the primary gas delivery, in which the primary gas entrains vapor or scent or liquid from the secondary agent, where the secondary agent has a purposeful effect to increase the effectiveness of the therapy, or to provide a secondary effect.

5. A device as in the claim 1 comprising a removable cartridge containing a secondary agent, positioned in the flow path of the primary gas delivery, in which the primary gas entrains the secondary agent, the agent selected from the group of: coagulant, oil, scenting agent, aroma therapy agent, lubricant, moisturizer, surface tension reduction agent to facilitate diffusion and penetration into the tissues, euphoric effect agent, a calming effect agent, awakening agent, a sleepy effect agent, a mind clarity agent, antimicrobial, antibacterial, antiviral, antifungal, disinfecting agent, numbing agent, anesthetic, analgesic, a reversing agent for a poison or for a drug, anti-inflammatory, steroid, neurotransmitter, stimulant, depressant, caffeine.

6. A device as in claim x, further wherein (a) the gas is a CO2 gas of 99.95% purity; (b) the gas cylinder is removable and replaceable from the device; (c) the nozzle is removably attachable from the device; (d) the pushing device is a spring powered pump; (e) the valve is gas flow channel with an open/closed button; and further comprising an actuator to release the spring from a compressed state.

7. A device as in claim 1 wherein an external gas cylinder is attachable to the device to provide additional volume of gas.

8. A device as in claim 1 wherein an external gas cylinder is attachable to the device to refill the internal gas cylinder.

9. A device as in claim 2 wherein the pushing mechanism is a pump.

10. A device as in claim 2 wherein the pushing mechanism is a syringe.

11. A device as in claim 1 wherein the gas is selected to mechanically cleanse the tissue structures.

12. A device as in claim 1 wherein the gas is selected to have a biological cleansing action by modulation of cell membrane activity.
